(19) United States
(12) Patent Application Publication
(10) Pub. No.: US 2026/0295179 A1
(43) Pub. Date: Oct. 1, 2026

(54) PRE-PRIMED GIVING SET AND COMPOUNDING APPARATUS' AND METHODS

(71) Applicant: TESSEN SOLUTIONS LIMITED, Carrigaline (IE)

(72) Inventors: Vincent FORDE, Carrigaline (IE); James ROCHE, Carrigaline (IE); Ciara WELDON, Carrigaline (IE)

(73) Assignee: TESSEN SOLUTIONS LIMITED, Carrigaline (IE)

(21) Appl. No.: 19/478,638

(22) PCT Filed: Apr. 25, 2024
(86) PCT No.: PCT/EP2024/061481

§ 371 Date: Oct. 24, 2025

(30) Foreign Application Priority Data
Apr. 27, 2023 (EP) 23170491.7

(51) Int. Cl. A61M 5/36; A61J 1/10; A61J 1/14; A61M 5/14; A61M 39/20; A61M 39/24

(52) U.S. Cl. CPC A61M 5/36; A61J 1/10; A61M 39/20; A61M 39/24; A61J 1/1475; A61M 2005/1402; A61M 2205/7536

(57) ABSTRACT

A method of preparing a giving set for administration of intravenous therapy to a patient, in a pharmacy. The method comprising steps of providing a filled IV bag (10) with an outlet port (12), connecting an IV line (20) to the outlet port, and connecting a gas trap device (30) to a distal end of the IV line. The trap device has a chamber (31) and a priming port (34) which is open. The method further includes squeezing the bag to force gas from the bag and through the line and out of the trap device. When liquid reaches the trap device (30) and substantially fills the chamber, the priming port is closed, thereby providing a pre-primed giving set comprising the bag, the line and the trap device filled with liquid. The actuator may have a roller which is forced in a direction towards the outlet port while the bag is constrained in a housing (51).

### INTRODUCTION

The present invention relates to prevention of gas moving into patient blood vessels during intravenous (“IV”) therapy.

The presence of gas in intravenous (IV) infusion sets administered to patients is an ongoing issue dealt with in hospitals and home care settings on a regular basis. It is imperative that as much gas as possible is removed from IV giving sets prior to use to prevent the occurrence of air emboli. Air embolism refers to the blockage of a vein or artery due to an air bubble present in the vascular system. Should this blockage travel to the heart or brain the consequences can be fatal.

The carrier fluid for IV administration is often a sterile saline solution to which other drugs may be added. A byproduct of the manufacturing and filling process is that a certain volume of gas remains in the IV bags containing the saline solution. The gas content may also increase as the IV bags are transported and a certain amount of gas becomes undissolved.

An IV bag is filled with liquid such as saline, blood or drugs. During manufacture involving filling of the bag, it is typically the case that an amount of gas is included in the bag before it is sealed, an unavoidable part of the filling process.

In many cases the process of removing air from IV lines starts at the pharmacy compounding stage. Compounding is the process of combining one or more drugs with the contents of the IV bag, therefore tailoring the medication to the specific patient/application. In this case, the drug(s) being added are the active pharmaceutical ingredient (API) and saline solution is typically the excipient. The API is added via syringe through the injection port of the bag or, in the case of hazardous drugs (HD) using a closed system transfer device (CSTD). The process of compounding can be quite time-consuming and physically taxing on the healthcare professional (HCP). The current method of air removal at this stage often involves the repeated use of a syringe by the HCP to remove all the air within the bag. This can lead to repetitive strain injury (RSI) and is an inefficient process.

It is known to use a roller device to squeeze a bag to assist drug delivery, as described in US2012/0197212 (Basso et al), U.S. Pat. No. 5,211,626 (Frank et al), and U.S. Pat. No. 5,728,077 (Williams et al).

It is known to provide gas traps to block passage of air and other gases in intravenous (“IV”) lines, examples being described in our published specifications WO2020/156666 and WO2022/023079, the contents of which are incorporated herein by reference.

The present invention is directed towards providing for enhanced infusion giving sets which provide a reduced risk of gas embolism. Another objective is to provide improved methods and apparatus of preparing giving sets, especially during compounding, to achieve simpler and more effective compounding and to minimise the risk of gas embolism in the prepared giving set.

### SUMMARY OF THE INVENTION

We describe a pre-primed giving set comprising:
an IV bag with liquid,
an outlet conduit connected to an outlet port of the bag, and
a gas trap device connected to the conduit, said device comprising:
   a gas trap chamber,
   a priming port which is configured to allow, during compounding, gas to escape and to be sealed when all gas has been expelled,
   a trap device outlet port for connection to a line for delivery of IV liquid to a patient,
wherein the bag, the line, and the trap device form a sealed system from which gas has been expelled.

In some preferred examples, the priming port comprises a backcheck valve. In some preferred examples, the priming port comprises a hydrophobic filter and a gas filter to neutralize gas toxic components. In some preferred examples, the priming port comprises in series and aligned in a direction away from the chamber, the hydrophobic filter, the gas filter, the backcheck valve, and a sealing cap.

In some preferred examples, the gas trap device comprises an inlet port linked with the bag, an outlet port, a gas trapping chamber between said ports, a diffuser coupled to the inlet port, and a diverter distally of the diffuser.

In some preferred examples, the priming port is in a wall of the chamber closer to the outlet port than the inlet port.

In some preferred examples, the giving set further comprises a second infusion line linking the gas trap device to a priming connector adapted to further expel gas and connect to a patient interface either directly or indirectly. In some preferred examples, the priming connector comprises a housing forming a flow chamber with axially arranged inlet and outlet ports, and a priming port in a side wall for radial outlet of gas.

In some preferred examples, said priming port comprises a hydrophobic filter, and laterally outwardly a gas filter, and further laterally outwardly a backcheck valve.

In some preferred examples, the priming connector further comprises a closed system transfer device connected to the outlet port of the priming connector.

In some preferred examples, the priming connector comprises a priming port axially in line with an outlet of the connector.

In some preferred examples, the giving set further comprises a third infusion line with a second gas trap device and said second infusion line is adapted to be coupled to a patient interface.

We also describe an apparatus for use in preparation of a giving set for example, the apparatus comprising a support for a bag and an actuator for squeezing the bag to force liquid from the bag and out through an outlet port of the bag until it reaches the gas trap device and gas is expelled via the priming port.

In some preferred examples, the apparatus further comprises a mount for allowing or causing rotation of a bag to an inverted orientation before operation of the actuator.

In some preferred examples, the apparatus comprises a bag housing and the housing is mounted for rotation while supporting a bag. Preferably, the apparatus comprises fasteners or tethers for engaging eyelets in top and bottom ends of a bag.

In some preferred examples, the actuator comprises a roller which is movable to squeeze a bag from a bottom end towards a top end, and the roller may be mounted across a pair of opposed slots, and the roller may advantageously be motor driven.

In some preferred examples, the actuator comprises an inflatable balloon or sleeve.

In some preferred examples, the apparatus further comprises a sensor to detect nature of bag and/or IV line contents, and a processor linked to the sensor to generate an output to indicate when gas has been expelled from the bag or line. The sensor may comprise an optical sensor. In some preferred examples, the processor is configured to stop operation of the actuator upon sensing that gas has been expelled from the bag or line.

We also describe a method of preparing a giving set for administration of intravenous therapy to a patient, the method comprising steps of:
providing a filled IV bag with an outlet port,
connecting an IV line to the outlet port,
connecting a gas trap device to a distal end of the IV line, the trap device having a chamber and a priming port which is open,
squeezing the bag to force gas from the bag and through the line and out of the trap device via the priming port,
when liquid reaches the trap device and substantially fills the chamber, closing the priming port, thereby providing a pre-primed giving set comprising the bag, the line and the trap device filled with liquid.

In some preferred examples, the trap device has a one-way valve in the priming port, allowing escape of gas but not gas ingress into the chamber. In some preferred examples, the bag is squeezed by an actuator. The actuator may comprise a roller which is forced in a direction towards the outlet port while the bag is constrained in a housing.

In some preferred examples, the method comprises the further steps of mounting the bag into a housing and rotating the housing to invert the bag, and subsequently operating an actuator to move to force gas from the bag through the line.

In various examples an apparatus of any of the above examples is used to squeeze the bag.

We also describe an apparatus for compounding and priming an IV bag, the apparatus comprising:
an injector device for injecting liquid medication via a liquid conduit into a bag for compounding, and
a gas removal device for removing gas from the bag via a gas conduit either during or after

In some preferred examples, the said gas removal device comprises a gas filter for cleaning of gas expelled from the bag as it is squeezed for priming.

In some preferred examples, the apparatus comprises a common needle providing one or more of said conduits for both liquid injection and gas expulsion.

In some preferred examples, the needle has only a single conduit, for sequential medication injection and gas expulsion whereby gas expulsion is possible only after compounding.

In some preferred examples, the needle comprises a dedicated gas conduit and a dedicated medication conduit or there are at least two needles (503, 504) each providing said dedicated conduits.

In some preferred examples, the injector device comprises a medication chamber for transfer of medication from a supply and a separate gas chamber for routing of expelled gas.

In some preferred examples, said chambers are linked by a backcheck valve.

In some preferred examples, the injector device has a bung for taking medication via a needle from a syringe.

In some preferred examples, the apparatus comprises a coupler for coupling to a medication vial for delivery of medication into the injector device.

In some preferred examples, the medication conduit projects outwardly in a supply direction to have an opening in a coupled vial.

In some preferred examples, the gas conduit projects outwardly in a supply direction to a greater extent than a medication conduit inlet or delivery of expelled gas into a top portion of a vial in use for pressure equalization as liquid medication is drawn from a lower level of the vial.

In some preferred examples, the gas conduit has an opening for outflow of gas via a filter and said opening for delivery of gas into a top portion of a vial.

In some preferred examples, the apparatus further comprises a hydrophobic filter between the gas filter and a gas conduit opening.

In some preferred examples, said gas conduit surrounds at least part of said medication conduit.

In some preferred examples, a gas inlet opening of the gas conduit is separated by a distance in the range of 1 mm to 30 mm from a liquid inlet of the medication conduit.

In some preferred examples, the apparatus further comprises a balloon to capture expelled gas.

We also describe a method of priming an IV bag using an apparatus of any example, the method comprising inserting the gas and medication conduits into the bag, connecting a medication supply to the apparatus, injecting medication via the medication conduit into the bag, and simultaneously or subsequently expelling gas from the bag via the gas conduit and directing the gas through the gas filter to ambient.

In some preferred examples, the medication is injected simultaneously with gas expulsion, and at least some of the expelled gas is delivered into a top portion of the vial for pressure equalization as medication flows via the medication conduit from the vial.

In some preferred examples, the apparatus comprising a needle for insertion into a bag, the needle being coupled with a suction pump for extraction of gas from a bag and delivering the gas into a chamber.

We describe an apparatus for use in preparation of an intravenous bag for a patient, the apparatus comprising a support for a bag and an actuator for squeezing the bag to force liquid from the bag and out through an outlet port of the bad and into a line.

In some examples, the apparatus further comprises a mount for allowing or causing rotation of a bag to an inverted orientation before operation of the actuator. In some examples, the apparatus comprises a bag housing and the housing is mounted for rotation while supporting a bag. In some examples, the apparatus comprises fasteners or tethers for engaging apertures in top and bottom ends of a bag. In some examples, the actuator comprises a roller which is movable to squeeze a bag from a bottom end towards a top end. In some examples, the roller is mounted across a pair of opposed slots. In some examples, the roller is motor driven.

In some examples, the apparatus further comprises a sensor to detect nature of bag and/or IV line contents, and a processor linked to the sensor to generate an output to indicate when gas has been expelled from the bag or line. In some examples, the sensor comprises an optical sensor.

In some examples, the processor is configured to stop operation of the actuator upon sensing that gas has been expelled from the bag or line.

We also describe a method of preparing a giving set for administration of intravenous therapy to a patient, the method comprising steps of:
providing a filled IV bag with an outlet port,
connecting an IV line to the outlet port,
connecting a gas trap device to a distal end of the IV line, the trap device having a chamber and a venting port which is open,
squeezing the bag to force gas from the bag and through the line and out of the trap device,
when liquid reaches the trap device and substantially fills the chamber, closing the venting port, thereby providing a pre-primed giving set comprising the bag, the line and the trap device filled with liquid.

In some examples, the trap device has a one-way valve in the venting port, allowing escape of gas but not gas ingress into the chamber. In some examples, the bag is squeezed by an actuator.

In some examples, the actuator is of an apparatus of any of example described herein.

In some examples, the actuator comprises a roller which is forced in a direction towards the outlet port while the bag is constrained in a housing. In some examples, the method comprises the further steps mounting the bag into a housing and rotating the housing to invert the bag and subsequently operating an actuator to move to force gas from the bag through the line.

We also describe a pre-primed giving set comprising an IV bag with liquid, and outlet line connected to an outlet port of the bag, and a gas trap device connected to a distal end of the line, in which the bag, the line, and the trap device form a sealed system from which gas has been expelled, and the trap device has a trap outlet port, which may include a closed system connector, for connection to a line for delivery of IV liquid to a patient.

### DETAILED DESCRIPTION OF THE INVENTION

The invention will be more clearly understood from the following description of some embodiments thereof, given by way of example only with reference to the accompanying drawings in which:

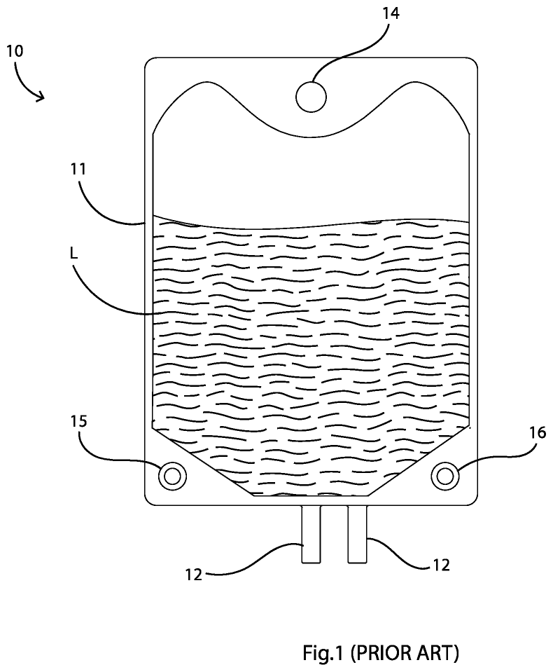

FIG. 1 is a front view of a typical infusion bag in its use orientation, with the ports lowermost for attachment to the IV line,

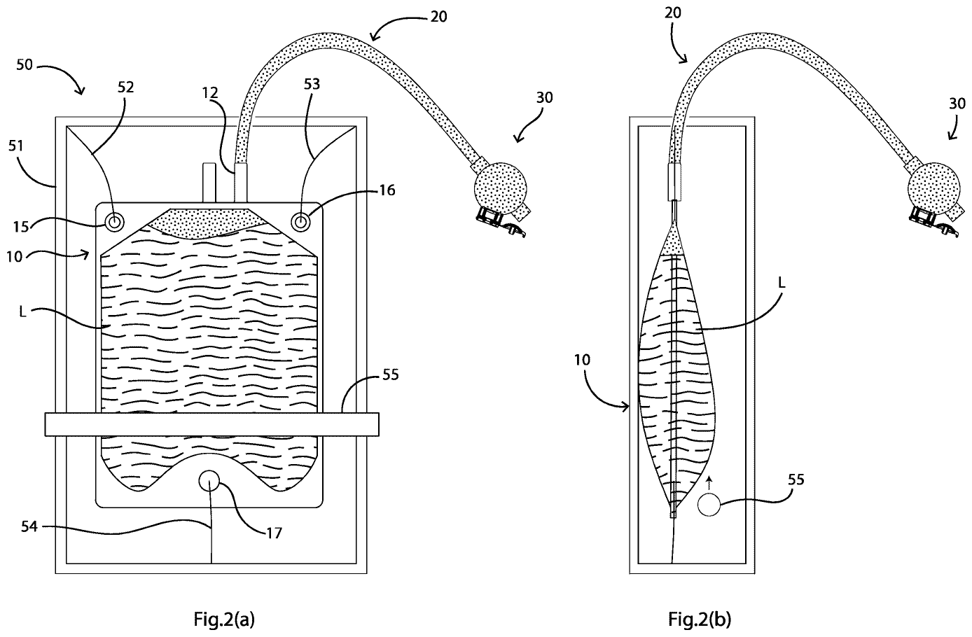

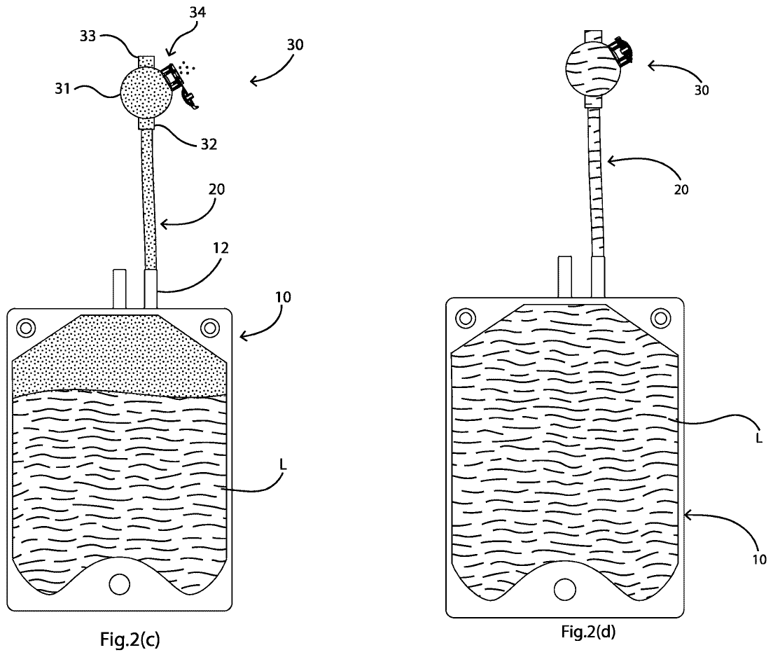

FIG. 2(a) is a diagrammatic front view showing a giving set and a gas expulsion apparatus of the invention, and FIG. 2(b) is a diagrammatic side view, FIG. 2(c) shows gas being expelled from the priming port of a gas trap device of the set, and FIG. 2(d) shows the giving set when all of the gas has been expelled and it is ready for use,

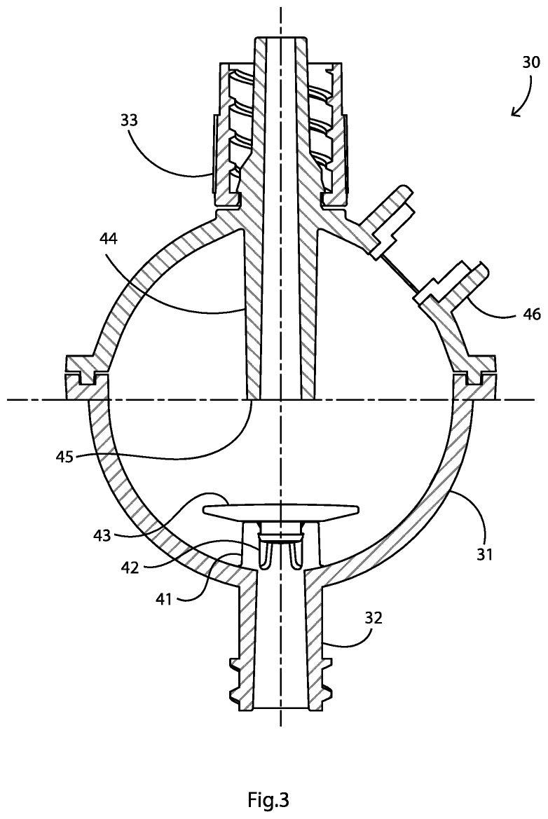

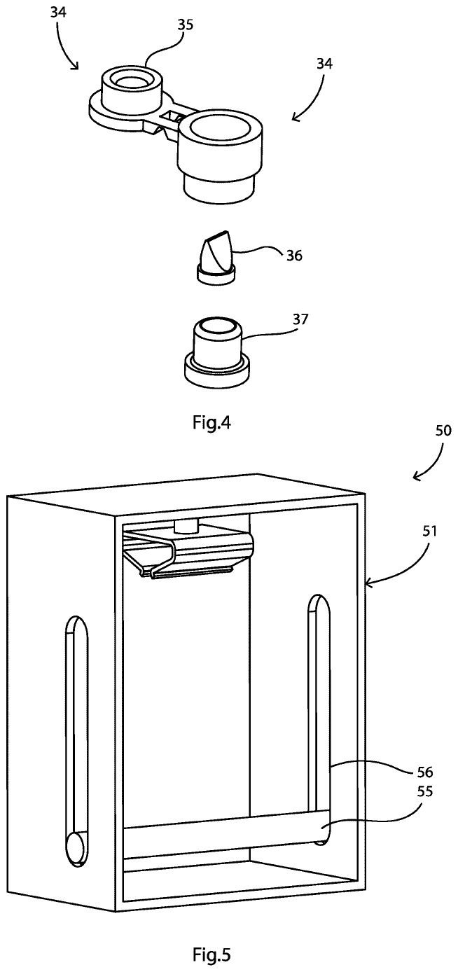

FIG. 3 is a cross sectional view of the gas trap device without the priming port, and FIG. 4 is an exploded view of the priming port,

FIG. 5 is a perspective view of the major components of a gas expulsion apparatus for use in preparation of the giving set,

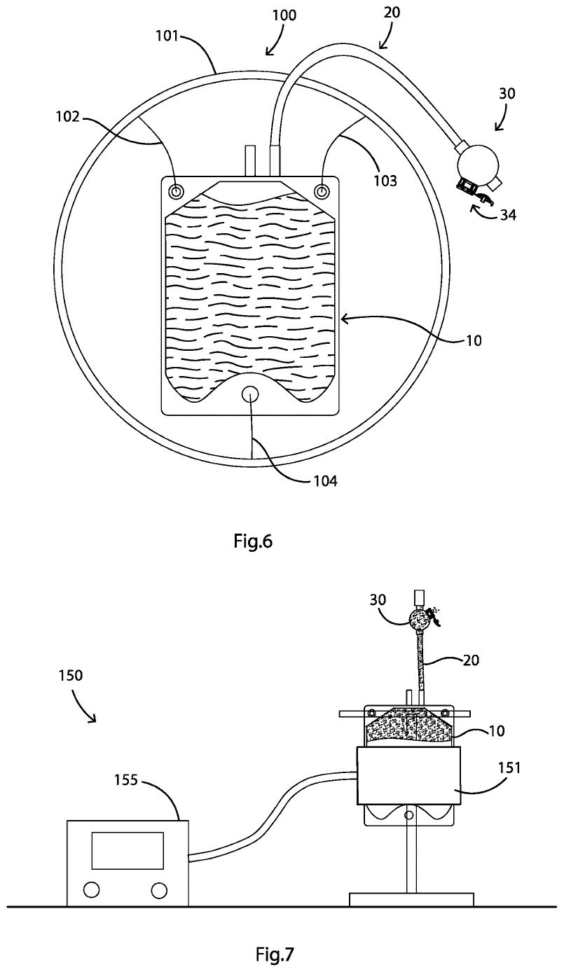

FIG. 6 is a diagram showing an alternative gas expulsion apparatus in use, which is rotatable,

FIG. 7 is a diagram showing a further alternative gas expulsion apparatus in use, in this case having an inflatable cuff for expelling gas from the IV bag,

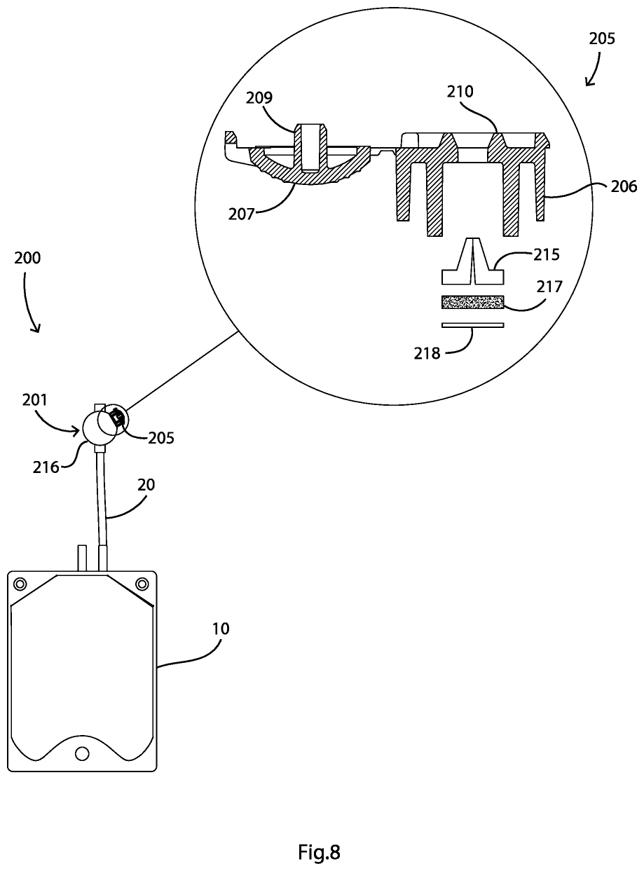

FIG. 8 shows an alternative priming port of a gas trap device of a giving set of the invention, in this case having a hydrophobic filter, a charcoal filter, and a backcheck valve,

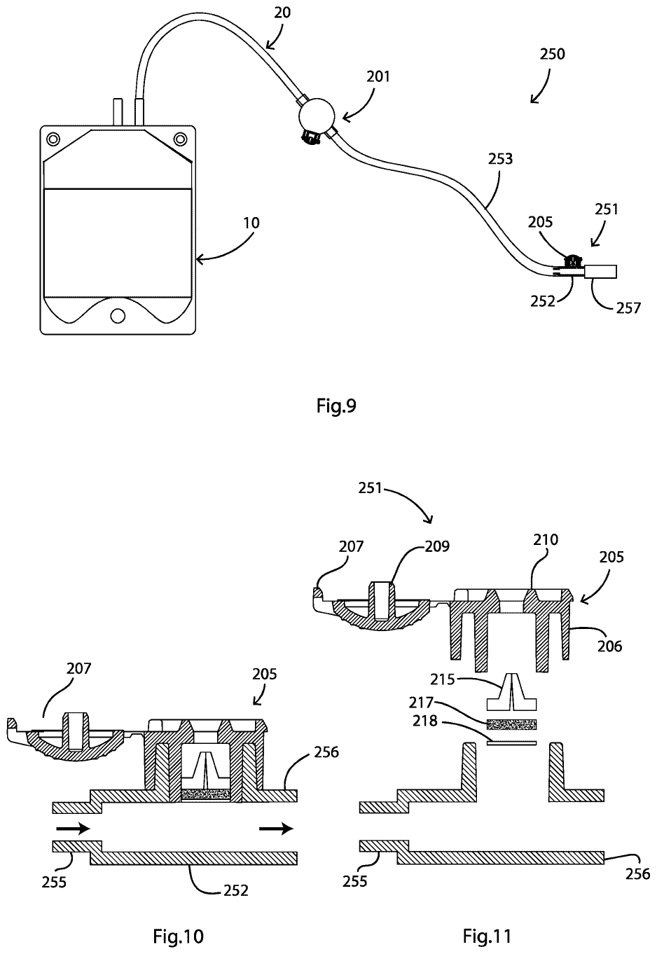

FIG. 9 shows an alternative giving set, in this case with a priming connector at a distal end,

FIG. 10 shows the priming connector in more detail, and FIG. 11 is an exploded view of the priming connector,

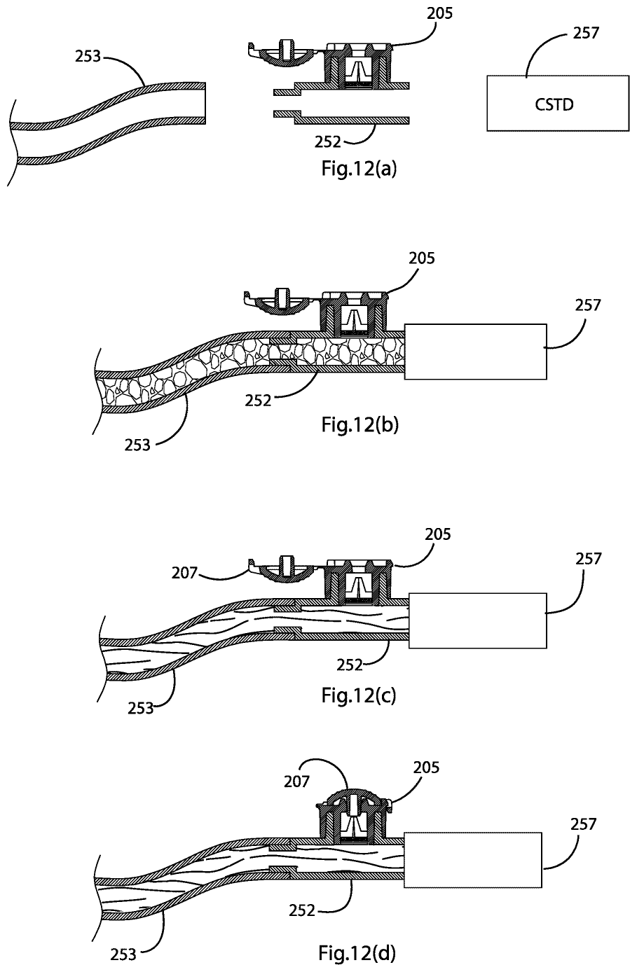

FIG. 12(a) to (d) inclusive show a series of steps for priming of this giving set,

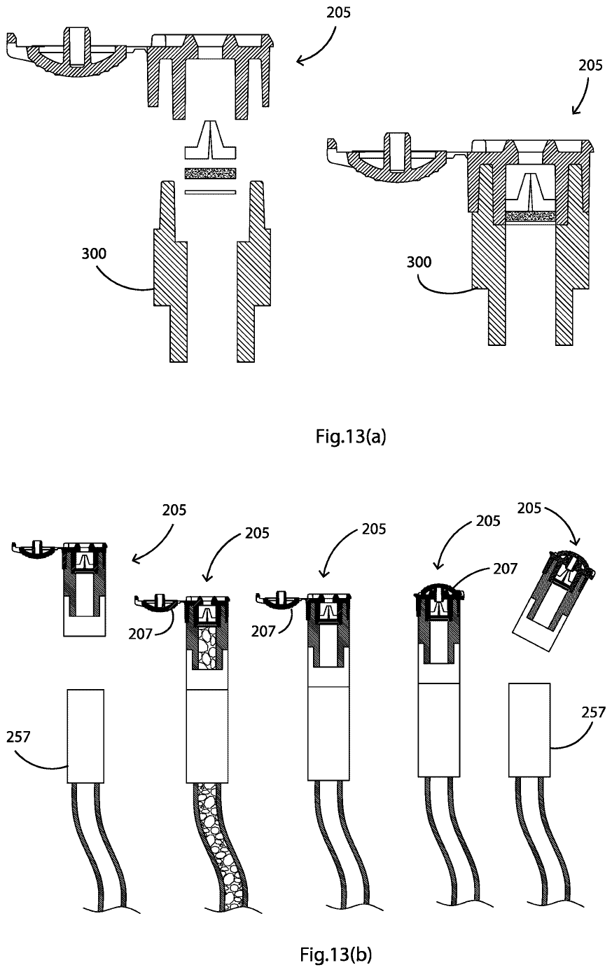

FIG. 13(a) shows the distal end of an alternative giving set, in this case with a priming connector with an inline priming port which is disposable, and FIG. 13(b) is a series of diagrams from left to right showing preparation of this giving set,

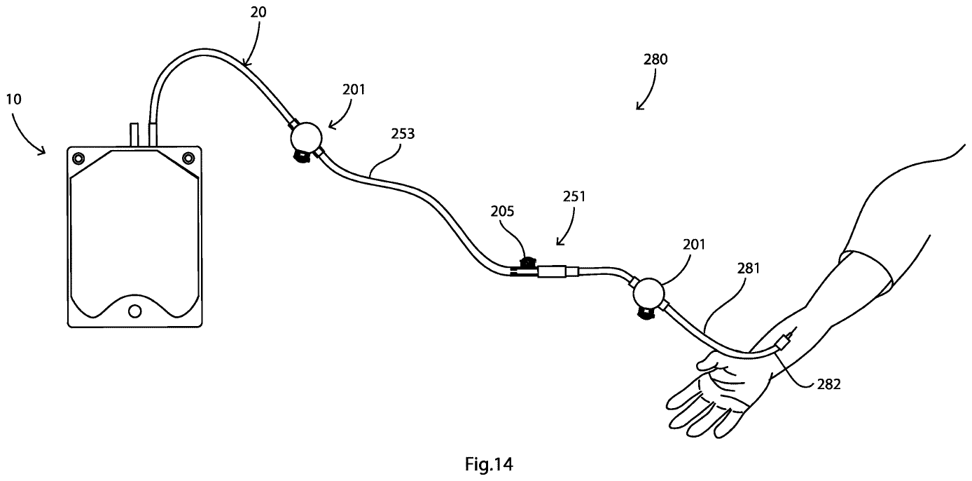

FIG. 14 is a perspective view of an alternative giving set of the invention, in this case with a second gas trap device,

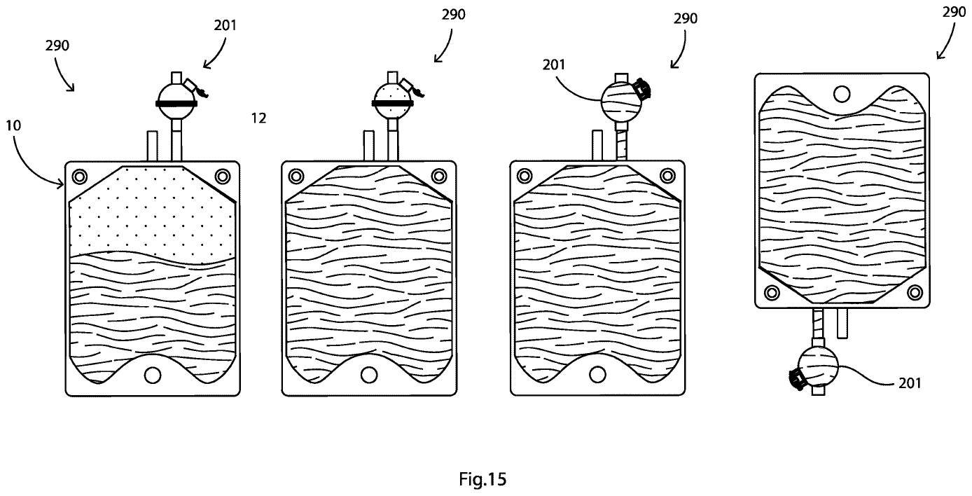

FIG. 15 is a series of diagrams showing priming of a further alternative giving set of the invention,

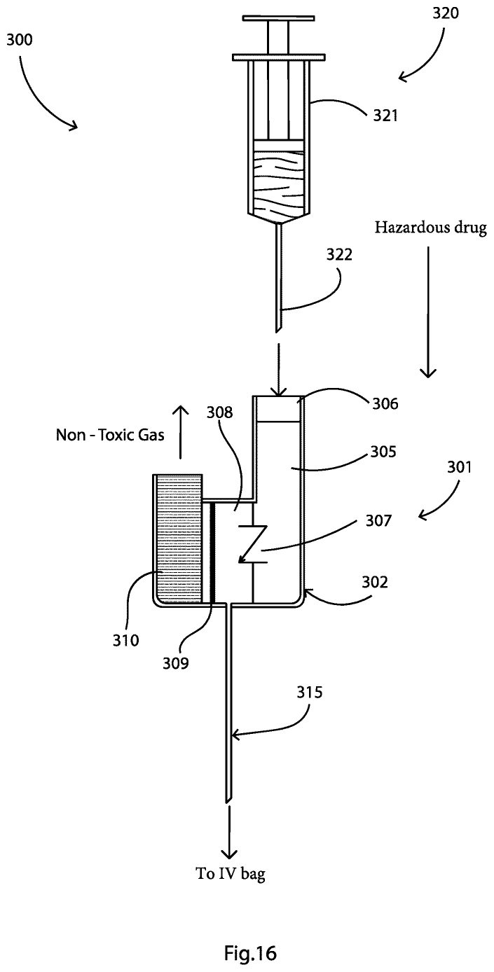

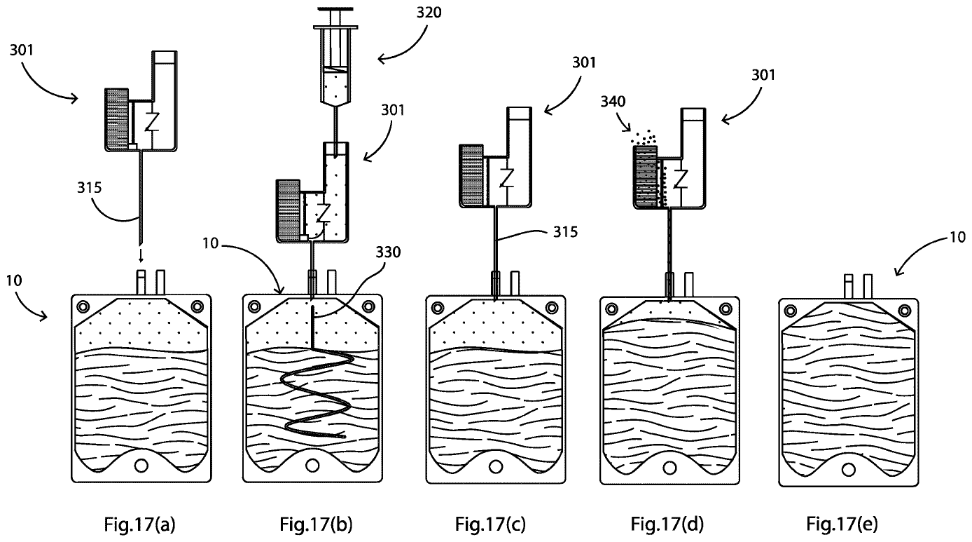

FIG. 16 is a sectional front view of a further gas expulsion apparatus, in this case for both gas expulsion and liquid drug injection, and FIG. 17(a) to (e) are diagrams showing a series of steps for use of this apparatus,

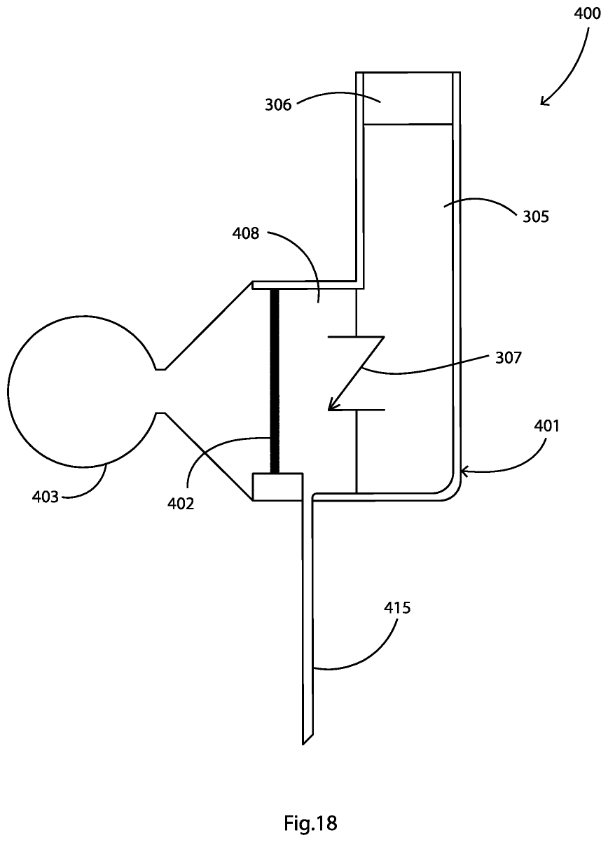

FIG. 18 is a sectional front view of an alternative apparatus for gas expulsion and drug injection, in this case having a balloon for trapping hazardous gases expelled from the bag,

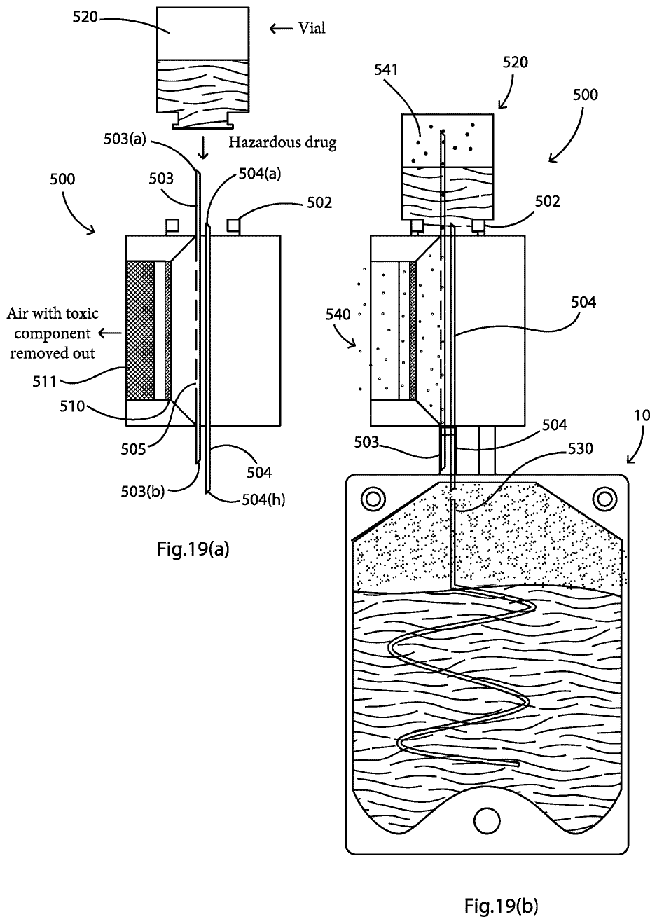

FIG. 19(a) and (b) are sectional front views showing use of an alternative apparatus for gas expulsion and drug injection in a manner which provides enhanced pressure balancing,

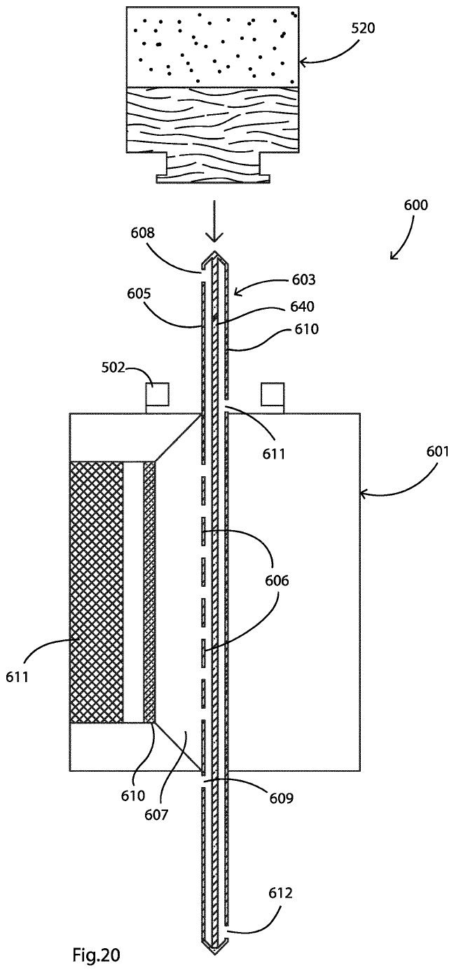

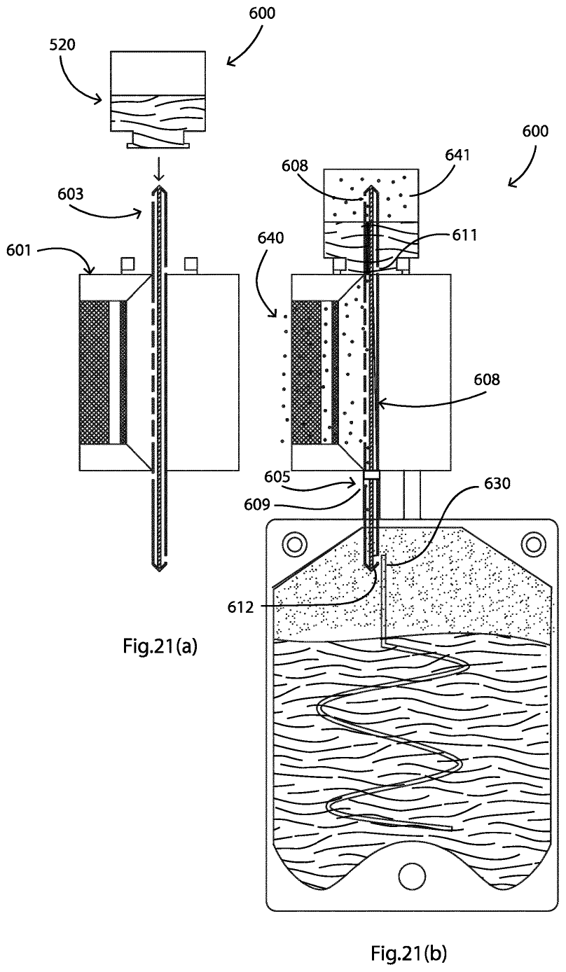

FIG. 20 is a sectional front view of a still further apparatus for both gas expulsion and drug injection with parallel gas and liquid conduits for contra flow in use, and FIGS. 21(a) and (b) show this apparatus in use,

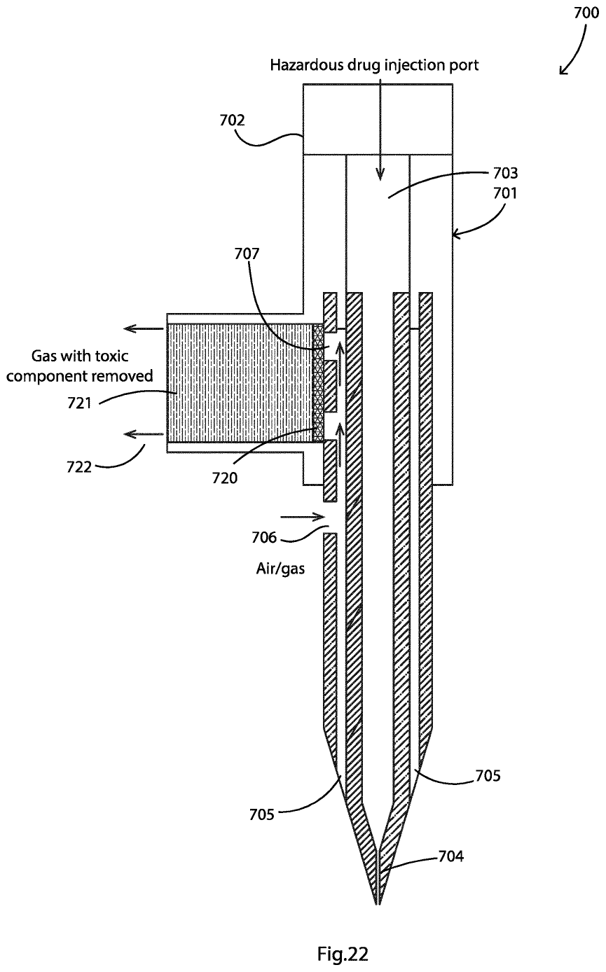

FIG. 22 is a sectional front view of a further apparatus, in this case having a central drug delivery conduit and an annular gas expulsion conduit, and

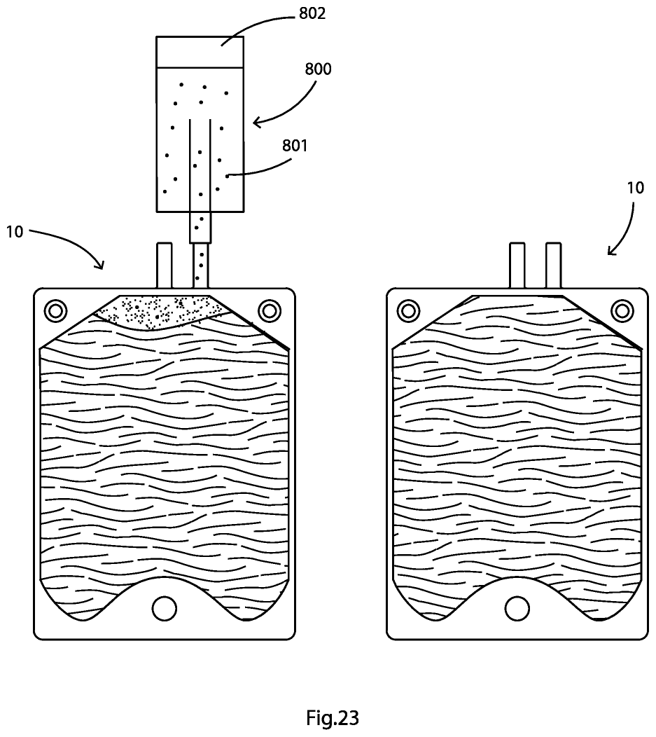

FIG. 23 is a pair of diagrams showing use of a gas expulsion apparatus with a suction pump for withdrawing gas into a chamber, and the primed bag on the right.

### DETAILED DESCRIPTION OF THE INVENTION

Referring to FIG. 1, an IV bag 10 has a bag body 11 with spikes 12 for delivery of the contents to an IV line and for injection of a drug. The bag 10 is conventional.

Referring also to FIG. 2(a) to (d) inclusive, in the pharmacy, preparation location or other supply centre, a pre-primed giving set of the invention is prepared using a gas expulsion apparatus 50. The giving set comprises the bag 10, an outlet line 20, and a gas trap device 30 connected together as shown in FIGS. 2 to 4. The bag 10 and the line 20 are themselves conventional, and the gas trap device is illustrated in most detail FIGS. 3 and 4. The connection of the bag 10, the line 20, and the trap device 30 together provide a giving set which is amenable to having gas expelled in a comprehensive manner so that it is ready for use with minimal risk of gas being delivered to a patient.

To effectively remove air from the bag 10, the bag 10 is inverted as shown in these drawings such that the ports 12 are facing upwardly and the gas is in the upper section of the bag 10. In this way, when the bag is spiked at the outlet port 12, the gas is positioned to vent out upwardly of the bag. Pressure is applied to the lower section of the bag, as indicated by the roller 55 in FIG. 2(b), pushing the liquid upwards and displacing the gas from the bag through the spiked port 12.

The action of forcing the liquid to cause the gas to be expelled is performed with the assistance of the apparatus 50 comprising a housing 51, and tethers 52, 53, and 54 supporting the bag 10 in an inverted orientation, at its holes 15, 16, and 17 respectively.

The gas trap device 30 is shown in more detail in FIG. 3 and has a spherical air trap chamber 31, an inlet port 32 linked to the line 20, an outlet port 33 which is closed, and a venting or priming port 34 shown in most detail in FIG. 4. The device 30 also has a diffuser 41 with apertures 42, and this is connected to a diverter 43. The device has an outlet conduit 44 with an opening 45 in the centre of the volume of the chamber 31.

As shown in FIG. 4 the priming port 34 has a main body to which is connected a cap 35, and within which is a one-way (backcheck) valve 36 in a valve housing 37. When the priming port 34 is open, the expelled gas coming down the line 20 under pressure can enter the device chamber 31 and is released through the open priming port 34.

The orientation shown in FIG. 3 is for preparation, in which the gas is expelled upwardly when the bag is inverted, and it escapes through the priming port 34 inserted in the rim 46 of the chamber 31. It will be appreciated that, after priming the priming port 34 is sealed and the port becomes part of the chamber. The device 30 has the opposite orientation in use with the liquid from the bag entering the inlet port 32 and flowing down through the diffuser and around the diverter 43. The diversion causes a laminar flow towards the internal surface of the chamber 31 and any remaining bubbles of gas are trapped in the upper part of the chamber near the diffuser 42, in a mechanism described in our published PCT specification number WO2020/156666.

By having the priming port 34 at the outlet end of the device 30 it is particularly suited for priming, and it does then not play any further role in use as it is sealed by the cap 35.

The pressure on the bag 10 continues until all of the gas is expelled from the bag, and liquid from the bag will then enter the IV line 20 and continue into the chamber 31 of the trap device 30, thereby priming the chamber 31 with liquid (FIG. 2(d)). Once filled with liquid, the pressure on the IV bag 10 is stopped and the priming port 34 is closed with the cap 35. Hence the giving set 60 made up of the bag 10, the line 20, and the device 30 are together a pre-primed giving set which is a closed system, as shown in FIG. 2(d). The device 30 performs a very useful function during preparation of the giving set and is then also during IV delivery to the patient. It will be noted that the priming port 34 is located on the in-use (IV delivery) outlet side of the device 30. This advantageously allows the device to be thoroughly primed as part of a giving set with the bag inverted (ports facing up).

In some scenarios, the outlet port 33 of the air trap device can have additional ‘extension’ tubing attached, which may be for connection to the patient interface.

The drug line extending from the trap device outlet port 33 can be attached to a patient and the drug is delivered from the bag to the patient via the in-line air trap 30, which acts as a reservoir for air. The administration of the IV treatment has the major benefits that there is little or no gas in the full giving set and moreover it is ready for use with a gas trap in place already primed. This reduces risk for treatment, especially for home care users.

The gas expulsion may be achieved by manual squeezing of the bag, with the benefit of not needing to use a syringe. However, it is preferred that an apparatus such as the apparatus 50 is used because it provides for squeezing the bag in a more controlled manner.

As shown in FIG. 5 the apparatus 50 has a housing 51 and the roller 55 which moves upwardly in slots 56 to squeeze the bag from the bottom up. The roller 55 runs in opposed vertical slots 56. The roller may be manually pushed up using handles at each end, or it may by urged up by a motor-driven actuator such as a lead screw or pneumatic piston.

However, in other examples the apparatus to squeeze the bag may comprise a vacuum, roller, cuff, or other mechanical actuator that reduces the internal volume of the bag, creating an internal pressure gradient that forces gas and liquid to be expelled under an increased pressure. The apparatus may have a hanger or other feature (such as the tethers in the apparatus 50) to enable the bag to be conveniently inverted and held in place, either through manual or automatic function. The device preferably provides very few steps required by the technician.

An apparatus for preparation of the pre-primed giving set may have a housing for the bag, and the housing preferably confines the bag to hold it in place to allow pressure to be applied by an actuator such as a roller. The housing may be rotatable, such that the bag can be hung and pivoted around a central axis to conveniently invert the bag. The housing may have clips or other fasteners to hold the bag in place. The housing may incorporate a clip to hold the air trap device in a desired location and to hold the IV line itself in place to reduce clutter. The bag may be spiked with an IV set, using a standard bag spike, Luer or closed system connector, or the bag may incorporate the IV set as a single entity. The apparatus may incorporate a sensor to alert the technician to the bag filling with liquid, the air being expelled, or the air trap device filling with liquid, or any scenario that indicates that the procedure is at or nearing a completion stage. The apparatus may incorporate a rack system and conveyor belt, such that several bags can be hung in place and automatically fed into the housing, providing hands-free, automated preparation of multiple IV bags. The apparatus may include a mechanism, such as a mechanically operated switch, to close the cap 35, providing further automation of the complete system.

FIG. 6 shows part of an alternative apparatus, 100, in this case having a housing 101 which is round in front view and has tethers 102, 103, and 104 for engaging a bag. This apparatus may have a roller which is pushed up, or any actuator such as any of the types mentioned above.

Referring to FIG. 7 an apparatus 150 is illustrated in which a pressure cuff 151 surrounds an IV bag 10 and forces entrained gas out via the IV line 20 to the gas trap device 30, the latter being as described above with the same reference numerals. This provides automated “burping” of the bag and there is no fluid contact between the apparatus 150 and the medication. This arrangement also prevents over-exertion of pressure due to suitable control of the inflation by a controller 155, in a manner akin to that of a blood pressure monitor controller. An apparatus with a cuff may additionally include any of the features of the apparatus 100 for rotating a bag to invert it before operation of the actuator.

In this example the actuator is a cuff surrounding a bag in use. However, it may in other embodiments comprise a balloon which is inflated to press only one side of the bag to force it against a hard surface on the opposite side in order to expel gas.

Additional gas-removal difficulties arise in compounding hazardous drugs (HD). In the preparation of HDs, the exposure of HCPs must be limited. The term “hazardous” is used to refer to a drug which has a qualitative level of toxicity, carcinogenicity, mutagenicity, and reproductive/developmental toxicity. The vast majority of HDs are anticancer chemotherapeutic agents known as antineoplastic drugs. At present, the compounding of antineoplastic drugs takes place under a fume hood to limit the inhalation of hazardous gases eluted during compounding. However, this is not an entirely safe method as hazardous gases may still make contact with the HCP.

The following describes further alternative giving sets which are advantageously used with a gas expulsion actuator of any embodiment. A giving set 200 is shown in FIG. 8. The giving set 200 includes a gas trap device 201 linked by a line 20 to a conventional bag 10. The device 201 is similar to the device 30 but the priming port is different, 205. The priming port 205 has a valve body 206 and a cap 207 which seals the port by insertion of a central tubular plug 209 into a socket 210. The port also includes a backcheck valve 215 to prevent gas from returning into the device's chamber 216, and beneath that there is a charcoal filter 217 and beneath that (closer to the chamber) a hydrophobic filter 218. The filter 17 may be any suitable adsorption filter/activated filter, such as activated carbon or charcoal. The combination, in direction radially out of the chamber 216, of the hydrophobic filter 218, the charcoal filter 217, and the backcheck valve 215 is particularly effective at preventing escape of HDs because it prevents ingress of gases and contaminants and prevents egress of liquid and gases. It is also advantageous that the port 205 is near the outlet side of the trap device, so that it is particularly effective for priming during preparation of the giving set. During use, the device is effective at preventing onward flow of gas bubbles by trapping them in the chamber.

The use of the giving set 200 allows the compounding of HD to take place in an ordinary lab setting or on the hospital ward as opposed to under a fume hood. It can be used to prevent contact with both HD in liquid form and the associated gases which elute.

Referring to FIGS. 9 to 11, a giving set 250 is illustrated and like parts are given the same reference numerals. In this case there is a bag 10, a first line 20, and a gas trap device 201 with a priming port 205 as described above. However, in addition there is a priming connector 251 at the distal end. The priming connector 251 is linked with the outlet port of the gas trap device 201 by a second line 253 and provides an alternative route for expelling gas from the set.

The priming connector 251 comprises an approximately cylindrical housing 252 that has the priming port 205 mounted radially as shown in FIGS. 10 and 11 with a single-use cap that is tamper-proof. The housing 252 has an inlet 255 coupled to the second line 253 and an outlet 256 coupled to a CSTD (closed system transfer device) 257. This priming port 205 has a combination of the hydrophobic filter 218, the carbon/charcoal filter 217, and the backcheck valve (BCV) 215. The purpose of the hydrophobic filter is to prevent escape of HD in liquid form through the venting port and prevents blocking of the carbon filter. The purpose of the carbon/charcoal filter is to scrub the hazardous gases of their toxic component. The purpose of the BCV is to prevent the re-introduction of air into the IV line.

The priming connector 251 is in use connected by its inlet 255 to the line 253 and by its outlet 256 to a closed system connector 257.

During priming, gas is forced through the priming port 205, which is placed at a 90° angle to the outlet direction such that the cap may be closed, and the connector 251 kept on the line following priming and for the duration of infusion. In more detail, FIG. 12 shows a series of four steps, from top down:
(a) Connection of the second line 253 to the priming connector inlet 255, and connection of the CSTD 257 to the outlet 256.
(b) Expelling of gas from the bag and along the lines via the gas trap device 201, with activation of the actuator. It is possible that this be done manually by manual squeezing of the bag.
(c) Expelling gas under this same pressure, out of the priming port 205 so that there is only liquid for all of the path to the CSTD 257.
(d) Closing the cap 207 of the priming port, so that the set 250 is sealed.

The priming connector is particularly advantageous if the plan were to limit manipulations of the IV line so that contamination is minimised. This means that the priming connector 251 would not need to be removed from the line and instead a suitable connection added to allow administration to the patient. This may also be done if the administration set is to be used directly after priming.

Referring to FIGS. 13(a) and (b) the priming port 205 may be mounted to the end of a line in an in-line manner downstream of a CSTD 257. In this case the priming port is solely used for the duration of the priming process and is removed prior to IV infusion. The in-line priming port 205 is placed directly across the path of fluid flow such that no gas can exit, thus protecting the HCP from HD in both liquid and gas form. This disposable device can then stay on the tubing until the primed administration set is given to the relevant HCP who can then safely disconnect the priming end cap, connect the desired set of IV tubing, and dispose of the device. The priming end cap also helps to prevent contamination of the administration set prior to infusion. FIG. 13(b) particularly shows the benefits, with, in series from left to right, the port 205 is added, used in expelling gas, sealed, and then removed.

When a set already has a CSTD connected at the end of the line, a mating CSTD can be connected to the proposed device. The device can then prime the tubing and be disconnected.

Referring to FIG. 14 an alternative embodiment is illustrated, a giving set 280 having the bag 10, the line 20, the trap device 201, the further line 253, the priming connector 251, and a further trap device 201 in a line 281 which is coupled at its distal end to the patient interface 282. This allows initial venting from the priming port f the first trap device 201, followed in turn by venting and sealing of the priming port 205 in the connector 251 and finally venting and sealing at the second (most distal from the bag) trap device 201.

FIG. 15 shows preparation of a simple giving set, 290, which comprises a bag 10 and a trap device 201 directly connected to the bag port 12. The gas is expelled with the bag 10 inverted, upon which the priming port is sealed, and the set is ready. Although this is a simple example, it has the benefit of the trap device allowing simple and effective expulsion of gas to prime the set, and then subsequent use when in its normal orientation to trap any remaining gas bubbles in its chamber 31. Again, the orientation of the priming port is facing away from the bag 10, at the distal end of the trap device 201.

The invention simplifies an existing manual process. Pharmacy technicians complain of carpal tunnel syndrome from repetitive strain of having to remove air from the bag using a syringe. Where the method is performed with an automated actuator physical exertion by the operator is not required. The gas trap device is a selectively sealable air trap, so it can be open during the ‘burping’ stage to release the air from the bag, then closed so the drug can be run. There is no need to remove the air trap device regardless of the drug being run.

By using an open filter to release air under pressure, and then closing the vent the method is very simple, where previously it required multiple steps.

As noted above a cabinet/housing can be used to assist squeezing of the bag (although this can be done by hand), but the cabinet can include useful features that make this easy to complete the burping—a sensor to detect if the air has been removed from the bag provides a convenient way of ensuring that liquid does not flow from the vent port. In some examples, the sensor activates a mechanism to close the cap, so the entire process is automated.

### Preparation/Compounding Apparatus With Combined Drug Injection and Gas Expulsion

FIGS. 16 to 22 illustrate examples apparatus of the invention which provide for both gas expulsion and effective delivery of a drug into the bag. These apparatus' can have an actuator such as described in any of FIG. 5, 6, or 7 or indeed they may simply have a support or housing to allow easy manual pressing of the bag. After use of the apparatus there is a primed IV bag with the desired medication ready for use. This primed bag may then be linked with a gas trap device, directly or indirectly, and the liquid may be pushed through the set until gas in the line and in the trap device is expelled through the priming port and the port is sealed, as described above. A difference is that the gases, which may be toxic, from the bag itself have already been removed and so the priming port of the gas trap device is only used to expel air in the line and the gas trap device when it has been connected to the pre-primed and prepared bag. In this case it may not be needed to have an active gas filter in the priming port, but one or both of a hydrophobic membrane and a backcheck valve are advantageous.

Referring to FIGS. 16 and 17 a bag preparation apparatus 300 has a contra-flow device 301 for downward flow of a liquid drug and parallel expulsion of gases from a bag. There is also a drug injection syringe 320 having a housing 321 and a needle 322 for injecting the drug into the device 301. The device 301 has a liquid chamber 305 and a bung 306 which is pierced by the needle 322 to inject the liquid drug into the chamber 305. This liquid flows through the chamber 305 and out through an outlet needle 315 to the bag in use. A backcheck valve 307 separates the liquid chamber 305 from a chamber 308 with a hydrophobic filter 309 and a charcoal filter 310 having a surface which is exposed.

As shown in FIG. 17(a) to (e) the liquid medication is injected from the syringe 320 prior to removal of air from the IV bag 10, so that the medication flows/drips down into the IV bag 10 in the inverted position via the chamber 305 and the needle 315. The backcheck valve (BCV) 307 prevents air from exiting via the inlet port. The other section of the device 301 contains the hydrophobic filter +carbon filter combination 309/310. Hence, following injection of the medication, the syringe 320 is removed, and the inlet is thus sealed by virtue of the rubber bung 306. The IV bag 10 can then be squeezed to remove all gas from the bag and, in doing so, scrub the gas of hazardous drug vapours via the filter combination 309/310. Hence a single connection to the bag 10 is used for initial drug delivery into the bag and then gas expulsion and filtering. This is very simple and effective.

The end product of use of the apparatus 300 is a primed and compounded bag 10 as shown in FIG. 17(e) and as noted above this may be used as it is or it may be connected to a line(s) and gas trap device(s) to provide a primed giving set as described above. Gas removal via the trap device priming port during the further preparation of the giving set does not involve release of hazardous gases because of the manner in which the bag has been primed, it is only essentially air in the line 20 and the trap device 201. Similar comments apply to the other apparatus' described in FIGS. 18 to 22 below.

Referring to FIG. 18 a dual device 400 has some parts in common with the device 301 and the same reference numerals are used. However, instead of venting cleaned gas to the surrounding atmosphere it is captured in a balloon 403 located on the outside of a hydrophobic filter 402 providing a gas exit from a chamber 408. A single needle 415 is provided for delivery of liquid medication into the bag and outflow of gas into the chamber 408 and into the balloon 403. An apparatus with a balloon for capturing expelled gas may also have a gas filter for cleaning gas before entry to the balloon to provide redundance in avoidance of expelling noxious gas to the environment.

Referring to FIGS. 19(a) and (b) an apparatus 500 is provided in which the liquid medication is introduced from a vial 520. The apparatus 500 has a resilient rim 502 for engaging the rim of the vial 520 when the vial is inverted. There is a needle-like channel 504 which extends downwardly from the apparatus housing and protrudes upwardly to a lesser extent. The channel 504 is open at both ends, with a top inlet end 504(a) for inflow of liquid and a bottom end 504(b) for outflow of this liquid. There is a parallel channel provided by another needle 503 which is also open at both ends, in this case a top end 503(a) for outflow of gas and a bottom end 503(b) for inflow of gas. The needle 503 protrudes upwardly to a greater extent than the needle 504, sufficient to extend into a top portion of the volume of the vial in use when it is engaged with the fixture 502. The conduit 503 also has pores 505 facing a chamber with a hydrophobic filter 510 and a charcoal filter 511.

As shown in FIG. 19(b) when the vial 520 is engaged with the apparatus the conduit 504 extends up into a lower portion of the vial and the conduit 503 extends into an upper portion of the vial volume. The apparatus 500 is akin to a syringe mechanism which travels between the vial 520 containing medication to be compounded and the IV bag 10. The apparatus 500 allows the medication in the vial to travel freely into the IV bag as shown diagrammatically by the stream 530. At the same time, or in a two-step process, the gas in the IV bag is directed through the hydrophobic +carbon filter combination for removal of the toxic component. In this case, the conduit 503 extends only into the space within the bag's injection port to allow for maximum removal of entrained gas. The term “dual lumen” may be used to refer to the syringe mechanism which has two channels through which fluids may flow. The orientation of these channels may differ depending on the application. One orientation involves the two channels running parallel to one another in a non-concentric fashion as illustrated.

It is very advantageous that the gas-flow syringe extends beyond the liquid-flow one to equalise pressure by allowing gas to flow from the IV bag into the top portion of the vial when the vial is inverted. The liquid-flow needle has a lower inlet opening (504(a)) to allow for maximum medication removal from the vial.

The syringe channel(s) on the inlet and outlet of the chamber may be both single lumens, both dual lumen or a combination of both types. The body of the device may also be flexible. The channels may be different sizes depending on the flow requirements of the liquid medication and the rate of gas removal.

Referring to FIGS. 20 and 21 an alternative dual channel apparatus, 600, is illustrated, again for use with a vial 520. The apparatus 600 has a dual conduit 603 having a central wall 640 to the right of which there is a liquid channel with a top inlet opening 611 and a lower outlet opening 612. The opposite side of the wall has a gas conduit with a lower gas inlet 609, pores 606, and a top gas outlet 608. The opening 609 is positioned so that in use it is within the port of the bag and in this case does not extend into the main body of the IV bag to allow for maximum gas removal. On this side the pores 606 open into a chamber 607 with a hydrophobic membrane 610 and a charcoal filter 611.

As shown in FIG. 21(b) in use the medication can flow out and down via the opening 612 as shown diagrammatically by the stream 630 while gases flow up and out into the ambient via the filters and up into the top volume of the vial for pressure balancing. The latter assists flow of liquid and traps these gases in the spent vial. FIGS. 20 and 21 are diagrammatic representations of how this type of dual channel apparatus would work. In practice, the dual conduit 603 may have a lesser diameter relative to the injection port of the bag.

In this embodiment, advantageously there is a syringe mechanism which travels between the vial containing medication to be compounded and the IV bag. This allows the medication in the vial to travel freely into the IV bag. At the same time, or in a two-step process, the gas in the IV bag is removed. This is directed through the hydrophobic+carbon filter combination for removal of the toxic component. Also, a portion of the gases travel into the upper part of the vial to equalize pressure. The pathway for gas to travel back up into the vial is largely for pressure equalisation as it is thought/predicted that the vast majority of the gas volume will be captured and released via the filter combination. The release of toxic gas from the vial is prevented by the rubber seal and metal or plastic cap which covers vials of hazardous drugs. The term “dual lumen” may be used to refer to such a syringe mechanism, which has two channels through which fluid may flow. The orientation of these channels may differ depending on the application. One possible orientation involves the two channels running parallel to one another in a non-concentric fashion, as in this example.

Referring to FIG. 22 an alternative apparatus 700 has a housing 701 with an upper bung 702 for reception of a needle from a syringe for medication injection. The housing 701 defines an inner conduit 703 for flow downward of medication and out via a lower opening 704 into a bag in use. There is an annular conduit 705 concentric with the conduit 703 and which is open at the lower end to receive gases from the bag. These travel up and exit via pores 707 into a filter combination of a hydrophobic membrane 720 and a charcoal filter 721. This is a particularly simple and effective apparatus, and the fact that the housing 701 is pointed at its lower end allows easy insertion through the port of the bag.

In this example, the lower inlet through which gas is removed 705 is in close proximity to the lower opening for ejection of liquid medication 704. This is a diagrammatic representation and in principle the distance between channels 705 and 704 may be larger. The distance above the liquid conduit that the gas conduit lower openings may be in the range of 1 mm to 30 mm, preferably 3 mm to 30 mm. These same parameter ranges also apply to the distance between medication inlet and gas outlet in a vial in use.

### Collection Apparatus

In these examples the apparatus operates to prepare a bag by automated suction of gases from the bag. Referring to FIG. 23 an apparatus 800 has a suction pump 801 which connects to a bag port and pulls gases out and up into a collection chamber 802. This is a crucial step in the pharmacy compounding process that is generally done by hand via a syringe and can lead to repetitive strain injuries. The automation of this device eliminates the manual element of syringe extraction.

This air-removal device is to be used on an inverted bag to, when active, suction air from the bag into a collection device. The collection device may be a pressurised vessel or expandable balloon and may have an its inlet a combination of hydrophobic filter+carbon filter+backcheck valve.

The air removal mechanism may be handheld or integrated into an overall compounding system. It may contain a sensor to detect when all the air has been removed from the bag.

Any of the apparatus of FIGS. 16 to 22 may additionally include a suction pump to assist gas evacuation and routing in a safe manner by filtering or capturing in a balloon.

Components of embodiments can be employed in other embodiments in a manner as would be understood by a person of ordinary skill in the art. The invention is not limited to the embodiments described but may be varied in construction and detail. For example, several embodiments include an “activated charcoal” filter. Carbon or charcoal filters become activated following exposure to either air at high temperature, carbon dioxide or water vapour. This significantly increases the surface are of carbon allowing for greater filtering capacity. This filter may take any form of known filter to neutralize toxicity, depending on the application. It may be an adsorptive organic gas filter, and is preferably disc shaped. It works due to a chemical reaction between the activated carbon molecules and the molecules to be removed which then become adsorbed onto the surface of the carbon filter. It may have bonded carbon which has a certain percentage of a binding agent. Depending on the width and thickness of the filter required, the binding material may be Polyethylene (PE) or glass fibre. Depending on the application, the width of the membrane may

1-49. (canceled)

50. A pre-primed giving set comprising:
an IV bag with liquid,
an outlet conduit connected to an outlet port of the bag, and
a gas trap device connected to the conduit, said device comprising:
a gas trap chamber,
a priming port which is configured to allow, during compounding, gas to escape and to be sealed when all gas has been expelled,
a trap device outlet port for connection to a line for delivery of IV liquid to a patient,
wherein the bag, the line, and the trap device form a sealed system from which gas has been expelled.

51. The pre-primed giving set as claimed in claim 50, wherein the priming port comprises a backcheck valve.

52. The pre-primed giving set as claimed in claim 50, wherein the priming port comprises a hydrophobic filter and a gas filter to neutralize gas toxic components.

53. The pre-primed giving set as claimed in claim 50, wherein the priming port comprises a hydrophobic filter and a gas filter to neutralize gas toxic components, and wherein the priming port comprises in series and aligned in a direction away from the chamber, the hydrophobic filter, the gas filter, the backcheck valve, and a sealing cap.

54. The pre-primed giving set as claimed in claim 50, wherein the gas trap device comprises an inlet port linked with the bag, an outlet port, a gas trapping chamber between said ports, a diffuser coupled to the inlet port, and a diverter distally of the diffuser.

55. The pre-primed giving set as claimed in claim 50, wherein the gas trap device comprises an inlet port linked with the bag, an outlet port, a gas trapping chamber between said ports, a diffuser coupled to the inlet port, and a diverter distally of the diffuser; and wherein the priming port is in a wall of the chamber closer to the outlet port than the inlet port.

56. The pre-primed giving set as claimed in claim 50, further comprising a second infusion line linking the gas trap device to a priming connector adapted to further expel gas and connect to a patient interface either directly or indirectly.

57. The pre-primed giving set as claimed in claim 50, further comprising a second infusion line linking the gas trap device to a priming connector adapted to further expel gas and connect to a patient interface either directly or indirectly; and wherein the priming connector comprises a housing forming a flow chamber with axially arranged inlet and outlet ports, and a priming port in a side wall for radial outlet of gas.

58. The pre-primed giving set as claimed in claim 50, further comprising a second infusion line linking the gas trap device to a priming connector adapted to further expel gas and connect to a patient interface either directly or indirectly; and wherein the priming connector comprises a housing forming a flow chamber with axially arranged inlet and outlet ports, and a priming port in a side wall for radial outlet of gas; and wherein said priming port comprises a hydrophobic filter, and laterally outwardly a gas filter, and further laterally outwardly a backcheck valve.

59. The pre-primed giving set as claimed in claim 50, further comprising a second infusion line linking the gas trap device to a priming connector adapted to further expel gas and connect to a patient interface either directly or indirectly; and wherein the priming connector further comprises a closed system transfer device connected to the outlet port of the priming connector.

60. The pre-primed giving set as claimed in claim 50, further comprising a second infusion line linking the gas trap device to a priming connector adapted to further expel gas and connect to a patient interface either directly or indirectly; and wherein the priming connector comprises a priming port axially in line with an outlet of the connector.

61. The pre-primed giving set as claimed in claim 50, further comprising a second infusion line linking the gas trap device to a priming connector adapted to further expel gas and connect to a patient interface either directly or indirectly; and wherein the giving set further comprises a third infusion line with a second gas trap device and said second infusion line is adapted to be coupled to a patient interface.

62. An apparatus for use in preparation of a giving set of claim 50, the apparatus comprising a support for a bag and an actuator for squeezing the bag to force liquid from the bag and out through an outlet port of the bag until it reaches the gas trap device and gas is expelled via the priming port.

63. The apparatus as claimed in claim 62, wherein the apparatus further comprises a mount for allowing or causing rotation of a bag to an inverted orientation before operation of the actuator; and wherein the apparatus comprises a bag housing and the housing is mounted for rotation while supporting a bag; and wherein the apparatus comprises fasteners or tethers for engaging eyelets in top and bottom ends of a bag.

64. The apparatus as claimed in claim 62 wherein the actuator comprises a roller which is movable to squeeze a bag from a bottom end towards a top end; and wherein the roller is mounted across a pair of opposed slots; and wherein the roller is motor driven.

65. The apparatus as claimed in claim 62, further comprising a sensor to detect nature of bag and/or IV line contents, and a processor linked to the sensor to generate an output to indicate when gas has been expelled from the bag or line; and wherein the sensor comprises an optical sensor; and, wherein the processor is configured to stop operation of the actuator upon sensing that gas has been expelled from the bag or line.

66. A method of preparing a giving set for administration of intravenous therapy to a patient, the method comprising steps of:
providing a filled IV bag with an outlet port,
connecting an IV line (20) to the outlet port,
connecting a gas trap device to a distal end of the IV line, the trap device having a chamber and a priming port which is open,
squeezing the bag to force gas from the bag and through the line and out of the trap device via the priming port,
when liquid reaches the trap device and substantially fills the chamber,
closing the priming port, thereby providing a pre-primed giving set comprising the bag, the line and the trap device filled with liquid.

67. The method as claimed in claim 66, wherein the trap device has a one-way valve in the priming port, allowing escape of gas but not gas ingress into the chamber.

68. The method as claimed in claim 66, wherein the bag is squeezed by an actuator.

69. The method as claimed in claim 66, wherein the bag is squeezed by an actuator; and wherein the actuator comprises a roller which is forced in a direction towards the outlet port while the bag is constrained in a housing.

70. The method as claimed in claims 66, comprising the further steps of mounting the bag into a housing and rotating the housing to invert the bag and subsequently operating an actuator to move to force gas from the bag through the line.

71. The method as claimed in claim 66, wherein the bag is squeezed by an apparatus comprising a support for a bag and an actuator for squeezing the bag to force liquid from the bag and out through an outlet port of the bag until it reaches the gas trap device and gas is expelled via the priming port.
